(19) United States
(12) Patent Application Publication
(10) Pub. No.: US 2013/0315982 A1
(43) Pub. Date: Nov. 28, 2013

(54) LIPOSOMAL DRUG COMPOSITION CONTAINING A POLYMERIC GUANIDINE DERIVATIVE

(71) Applicants: Oskar Schmidt, Wien (AT); Andreas Wagner, Wien (AT); Barbara Rupp-Stanschitz, Klein-Jetzelsdorf (AT)

(72) Inventors: Oskar Schmidt, Wien (AT); Andreas Wagner, Wien (AT); Barbara Rupp-Stanschitz, Klein-Jetzelsdorf (AT)

(73) Assignee: MINDINVEST HOLDINGS LTD., Valletta VLT (MT)

(21) Appl. No.: 13/882,344

(22) PCT Filed: Oct. 28, 2011
(86) PCT No.: PCT/EP2011/005453

§ 371 Date: Aug. 9, 2013

(30) Foreign Application Priority Data
Oct. 29, 2010 (AT) A 1796/2010

(51) Int. Cl. A61K 9/127; A61K 31/155

(52) U.S. Cl. CPC A61K 9/127; A61K 31/155
USPC 424/450; 514/634

(57) ABSTRACT

A liposomal drug composition comprising: a dimeric or polymeric guanidine derivative or a pharmaceutically acceptable salt thereof as drug substance, and a lipid modified by polyethylene glycole (PEG). The drug composition have cytostatic and antimicrobial activity.

The invention relates to a drug composition containing a dimeric or polymeric guanidine derivative.

It is known e.g. from EP 1 597 302 B1 and EP 1 605 927 B1 that polymeric guanidine derivatives display a strong antimicrobial activity and also cytostatic activity and that these derivatives can be used as drug substances to formulate drug compositions.

The inventors of the of the present invention observed that polymeric guanidine derivatives—when applied intravenously—tend to attach to blood vessels and thus diminishing the amount of drug substance to reach the tumor cells and the place of the microbial infection. Further, attachment to blood vessels, e.g. affinity to endothelial cells, may cause damage to the vessels and the cells while not reaching the tumor cells in sufficient concentrations. The present invention aimes at overcoming this problem.

It has been shown that the problem can be solved by encapsulation of the polymeric guanidine derivate in PEGylated liposomes, which ensures that the drug substance reaches its target in higher concentrations, e.g. the malignant tumor cells. In addition, the inventive drug composition presents therapeutic advantages due to the fact that the API (active pharmaceutical ingredient) can kill tumor cells without inducing resistance (a resistance study with 30 passages has shown no reduction of activity). The commonly known cytostatics induce such cellular resistance mechanisms requiring higher, toxic doses, which cause severe side effects. With the drug composition according to the present invention the therapy can be conducted on a daily basis with a relatively low dose allowing the active substance to be absorbed by the tumor cells, which consequently are being completely inhibited, without causing severe systemic side effects.

The present invention therefore is directed to a liposomal drug composition comprising: a dimeric or polymeric guanidine derivative or a pharmaceutically acceptable salt thereof as drug substance, and

a lipid modified by polyethylene glycole (PEG), i.e. a PEGylated lipid.

The lipid preferably is a phospholipid and said PEG is PEG₅₀₀-PEG₅₀₀₀.

A preferred embodiment of the liposomal drug composition according to the invention is characterized in that said polymeric guanidine derivative is one, which guanidine derivative is based on a diamine containing oxyalkylene chains between two amino groups, with the guanidine derivative representing a product of polycondensation between a guanidine acid addition salt and a diamine containing polyoxyalkylene chains between two amino groups.

Among the representatives of the family of polyoxyalkylene guanidine salts, there are preferred such using triethylene glycol diamine (relative molecular mass: 148), polyoxypropylene diamine (relative molecular mass: 230) as well as polyoxyethylene diamine (relative molecular mass: 600).

Preferred is also a liposomal drug composition, characterized in that poly-[2-(2-ethoxyethoxyethyl)guanidinium hydrochloride] comprising at least 3 guanidinium groups is contained as the drug substance.

The average molecular mass of the drug substance can be from 500 to 3000.

The present invention is also directed to the use of a dimeric or polymeric guanidine derivative as defined above for the preparation of a cytostatically active liposomal drug composition.

Further, the present invention is directed to the use of a dimeric or polymeric guanidine derivative as defined above for the preparation of an antimicrobial drug composition.

A still further aspect of the present invention is a process for therapeutically treating human beings and animals, characterized in that a drug composition according to the present invention as defined above is injected into a human being or an animal in need thereof.

The liposomes were prepared according to the method, which is described in detail in EP 1 337 322 and U.S. Pat. No. 6,843,942.

The liposome preparation method can be described as modified ethanol injection system. Liposomes are produced by the crossflow injection technique which is a highly reproducible technology for the active and/or passive incorporation of a variety of pharmaceutical active substances into liposomes. Continuous aseptic one step operation permits to produce stable and sterile liposomes with defined size distribution. The production equipment is designed to meet several requirements such as simplicity, ruggedness and easy handling in sterilization procedures.

In brief, the lipid components, in particular DMPC, DPPC, DMPG, DSPE-PEG-2000 and cholesterol, are dissolved in a water miscible organic solvent, especially ethanol. The polymeric guanidin derivative, preferably poly-[2-(2-ethoxyethoxyethyl)guanidinium hydrochloride], is suspended in PBS or in physiological sodium chloride solution. The aqueous phases are either kept at 55° C. or at room temperature. The injection module, wherein the solvent and the aqueous phases are mixed, is equipped with an injection hole of 350 μm diameter. The lipid solution is merged with the aqueous active ingredient solution at an injection pressure of 5 bar and a flow rate of the aqueous phase between 200-500 ml/min. Liposome size and homogeneity can be controlled by the local lipid concentration at the injection/mixing point. The local lipid concentration is influenced by the lipid concentration in the organic solvent, the injection pressure, the injection bore diameter and the flow rate of the aqueous phase. Additional influence on the liposome size have the process temperature, the ionic strength of the aqueous phase and the osmolality of the chosen buffer system. Subsequent filtration steps are performed to remove untrapped API and residual organic solvent.

A flow chart describing the liposome formulation process is depicted in FIG. 1. In FIG. 1 the numbers indicated designate
(1) lipid ethanol solution
(2) aqueous phase
(3) aqueous phase 2
(4) API in liposomes
(5) aqueous phase+API
(6) waste
(7) final suspension
(8) ultra/dia' filtration

Standard liposomes, consisting of phospholipids and cholesterol, can be prepared with the procedure described above. Typical formulations contain DMPC and cholesterol, DMPC, DMPG and cholesterol, DMPC, DPPC and cholesterol or pure DPPC. After pre-formulation of the liposomes with the above standard method stepwise downsizing of the liposomes can be accomplished by extrusion through straight pore polycarbonate filters. Liposomes formulated and downsized in the presence of polymeric guanidine derivates as described tend to form larger structures than in the absence of these substances. Exemplary data are presented in Table 1 below.

TABLE 1

| | Lipids [μmol/ml] | | | | Size* | |
|---|---|---|---|---|---|---|
| | DMPC | Chol | DMPG | DPPC | [nm] | PdI |
| Empty liposomes | 15 | 1 | 1 | | 170.1 | 0.350 |
| Liposomes containing poly-[2-(2- | 15 | 1 | 1 | | >20000* | 0.550* |
| ethoxyethoxyethyl)guanidinium | | | | | | |
| hydrochloride] | | | | | | |
| Empty liposomes | 5 | 2 | | 10 | 146.7 | 0.206 |
| Liposomes containing poly-[2-(2- | 5 | 2 | | 10 | 381.9 | 0.603 |
| ethoxyethoxyethyl)guanidinium | | | | | | |
| hydrochloride] | | | | | | |
| Empty liposomes | 16 | 1 | | | 68.1 | 0.082 |
| Liposomes containing poly-[2-(2- | 17 | | | | 491.2* | 0.596* |
| ethoxyethoxyethyl)guanidinium | | | | | | |
| hydrochloride] | | | | | | |

Table 1 shows a comparison of empty liposomes and liposomes conaining poly-[2-(2-ethoxyethoxyethyl) guanidinium hydrochloride].
*Liposome size values underlined indicate poor quality data due to the inhomogeneity of these large and heterogeneous structures.

Two different formulations, namely DMPC and cholesterol and DMPC, DMPG and cholesterol, were chosen for additional studies on the effect of polymeric guanidin derivatives on the size of the formed liposomes using standard lipid compositions and standard processes. For these studies, empty as well as poly-[2-(2-ethoxyethoxyethyl)guanidinim hydrochloride] encapsulated liposomes were produced. A summary of these batches is presented in Table 2 below.

TABLE 2

| | | z-average | | Zeta | | |
|---|---|---|---|---|---|---|
| | | mean | | potential | | Osmolality |
| Batch | Composition | [nm] | PdI | [mV] | pH | [mOsm/kg] |
| #1 | DMPC/Chol + API | 1931 | 0.989 | 0.902 | 7.41 | 670.0 |
| #2 | DMPC/Chol − empty liposomes | 123.1 | 0.079 | −0.022 | 7.40 | 695.3 |
| #3 | DMPC/DMPG/Chol + API | too | | 0.072 | 7.39 | 1252.0 |
| | | inhomogeneous | | | | |
| #4 | DMPC/DMPG/Chol − empty | 124.8 | 0.083 | −9.81 | 7.41 | 1098.3 |
| | liposomes | | | | | |

Additional experiments were performed with the empty liposome batches in presence of free poly-[2-(2-ethoxyethoxyethyl)guanidinim hydrochloride]. In these experiments, the empty liposome samples were spiked with poly-[2-(2-ethoxyethoxyethyl)guanidinim hydrochloride] and after 1 h incubation, the samples were analysed with respect to liposome size, size distribution and zeta potential as summarized in Table 3 below.

TABLE 3

| | Original formulation | | | Spiked formulation | | |
|---|---|---|---|---|---|---|
| batch | size | PdI | zeta pot. | size | PdI | zeta pot. |
| #2 | 123.1 nm | 0.079 | −0.022 mV | 157.9 nm | 0.083 | 3.85 mV |
| #4 | 124.8 nm | 0.083 | −9.81 mV | 1234 nm* | 0.470* | −3.13 mV |

*poor quality data

Table 3 shows a summary of analytical data of empty liposomes before and after addition of free poly-[2-(2-ethoxyethoxyethyl)guanidinim hydrochloride].

The addition of the positively charged free poly-[2-(2-ethoxyethoxyethyl)guanidinim hydrochloride] changed the zeta potential of both liposome suspensions markedly. The suspension #2, which was composed of DMPC and cholesterol changed from a neutral/slightly negative surface potential to a positive, whereas the originally negatively surface potential of the suspension #4 was decreased by 6 mV to −3.13 mV. The addition of polyguanidin derivates also influences the hydrodynamic radius of the liposomes, which is measured by dynamic light scattering. The result for the particle size is given by the z-average mean and for the homogeneity by the polydispersity index. The “neutral” suspension shows an increase in vesicle size by approx. 30 nm, which can be related to membrane surface attached polyguanidin derivates. The negatively charged suspension #4 is stronger influenced by the addition of free positively charged polyguanidines. The former homogeneously distributed liposomes turn into large aggregates, which cannot be determined by the standard size measurements. This strong tendency to form aggregates can be explained by the interaction between the negatively surface charge of the liposomes and the positively charged polymeric API.

On the contrary according to the present invention liposomal formulations of polymeric guanidin derivates with PEGylated lipids do not form larger structures and/or aggregates.

Among other applications the present invention should be used in oncology. For this purpose passive tumor targeting after prolonged circulation in the blood stream should be achieved. As published in scientific literature passive targeting can be accomplished by introducing PEG-chains in the formulation of the drug.

In formulation experiments a mixture consisting of DMPC and the pegylated lipid DSPE-PEG-2000 was investigated. Compared to the first approach, where polymeric guanidin derivates were encapsulated in liposomes composed of standard phospholipids, there is no influence of the API on the formulation behavior of PEG-liposomes. Liposomes prepared in presence and absence of polymeric guanidin derivates do not differ in size range and homogeneity as set out in Table 4 below.

TABLE 4

| | | z-average | | Zeta | | |
|---|---|---|---|---|---|---|
| | | mean | | potential | | Osmolality |
| Batch | Composition | [nm] | PdI | [mV] | pH | [mOsm/kg] |
| #1 | DSPE-PEG-2000/DMPC + | 116.2 | 0.155 | −0.086 | 7.39 | 549.6 |
| | API | | | | | |
| #2 | DSPE-PEG-2000/DMPC − | 104.0 | 0.224 | −0.192 | 7.38 | 707.3 |
| | empty liposomes | | | | | |

Table 4 shows a summary of analytical data of empty PEGylated liposomes and PEGylated liposomes containing an API according to the present invention.

In addition, the API concentration has no influence on formulation behavior: As shown in Table 5 below, API concentration did not influence liposome size and homogeneity.

TABLE 5

| | Lipids | | | |
|---|---|---|---|---|
| | [μmol/ml] | | Size | |
| | DMPC | mPEG2000 | [nm] | PdI |
| PBS with 4% API | 21.375 | 1.125 | 103.4 | 0.182 |
| 25% API in 0.92% NaCl | 21.375 | 1.125 | 101.9 | 0.121 |
| 50% API in 0.92% NaCl | 21.375 | 1.125 | 103.7 | 0.142 |

Table 5 shows the influence of the API concentration on liposome size and homogeneity.

To investigate reproducability of the process according to the present invention a set of three batches was produced. The process parameters are given in Table 6 below. As shown in Table 7 below, all three batches are similar with respect to all tested parameters. This data accentuates the novelty of the present invention regarding reproducability, size distribution, homogeneity and inclusion rates of the API.

TABLE 6

| Aqueous phases + | 80 ml injection buffer |
|---|---|
| volumes | (39% API in |
| | phys. NaCl) |
| | 320 ml dilution |
| | buffer (PBS) |
| Injection module diameter | 350 μm |
| Ethanol concentration in intermediate liposome | 5% |
| suspension | |
| Injection pressure | 5 bar |
| Lipid concentration in intermediate solution | 28.5/1.5 |
| DMPC/DSPE-PG-2000 [μmol/ml] | |
| API concentration | 39% |
| Temperature ethanol solution/aqueous phase | 55° C. |
| Diafiltration | 15-20 volume changes |
| Ultrafiltration: concentration factor | 4 |

Table 6 shows the process parameters.

TABLE 7

| | Size | | Zeta pot. | | Osmolality | API |
|---|---|---|---|---|---|---|
| | [nm] | PdI | [mV] | pH | [mOsm/kg] | [mg/mL] |
| Batch 1 | 121.4 | 0.143 | −0.218 | 7.41 | 289 | 15.8 |
| Batch 2 | 123.9 | 0.178 | −0.241 | 7.45 | 305 | 14.8 |
| Batch 3 | 124.5 | 0.137 | −0.039 | 7.49 | 312 | 12.4 |

Table 7 shows analytical data of three reproducability batches.

Using standard in-vitro cell culture tests it could be shown that a liposomal formulation of the guanidine polymer according to the present invention is highly active against several malignant cell lines.

The activity of different concentrations of a liposomal formulation according to the present invention on several cell lines have been tested using an H-thymidin-test. The activity on the following cell lines has been studied:
1) Hematological malignant cell lines:
   a. Myeloic: HL60, K 562
   b. Lymphatic: CEM C7H2, U937
2) Malignant solid cell lines:
   a. Prostate carcinoma (DU 145)
   b. Non Small Cell Lung Cancer (A-549)
   c. Ovarian carcinoma (OVCAR-3)
   d. Breast cancer (ZR-75-1)
   e. 2 Glioblastoma cell lines (U-373 and T98-G)

The growth of acute myeloic and lymphatic cell lines has been inhibited up to 50% by 2.5 μM and up to 100% by 5 μM of the liposomal formulation according to the present invention. The chronic myeloic and the lymphatic cell line U-937 reacted even more sensitive and showed a 100% growth inhibition at 2.5 μM.

As shown in FIG. 3, all tested solid carcinoma cell lines have been inhibited to 100% at a concentration of 5 μm. Investigations indicate that the non small cell lung cancer cell lines were the most sensitive of all tested cell lines to the liposomal formulation according to the present invention and show nearly complete growth inhibition at 2.5 μM.

### BRIEF DESCRIPTION OF THE FIGURES

FIG. 1 shows a flow chart describing the liposome formulation process.

FIG. 2 to 11 show growth inhibition of liposomal formulation according to the present invention on several malignant cell lines, in detail:

FIG. 2 shows inhibition of growth of HL 60 cell lines (acute promyelocyte leukemia)

FIG. 3 shows inhibition of growth of CEM cell lines (T-cell acute lymphoblastic leukemia)

FIG. 4 shows inhibition of growth of K-562 cell lines (chronic myelogenous leukemia)

FIG. 5 shows inhibition of growth of U-937 cell lines (anaplastic diffuse large B-cell lymphoma)

FIG. 6 shows inhibition of growth of DU-145 cell lines (prostatacarcinom)

FIG. 7 shows inhibition of growth of A-549 cell lines (non-small cell lung carcinoma)

FIG. 8 shows inhibition of growth of OVCAR-3 cell lines (ovary adenocarcinoma)

FIG. 9 shows inhibition of growth of ZR-75-1 cell lines (mamma carcinoma)

FIG. 10 shows inhibition of growth of U-373 cell lines (glioblastoma multiforme), and

FIG. 11 shows inhibition of growth of T-98-G cell lines (glioblastoma multiforme)

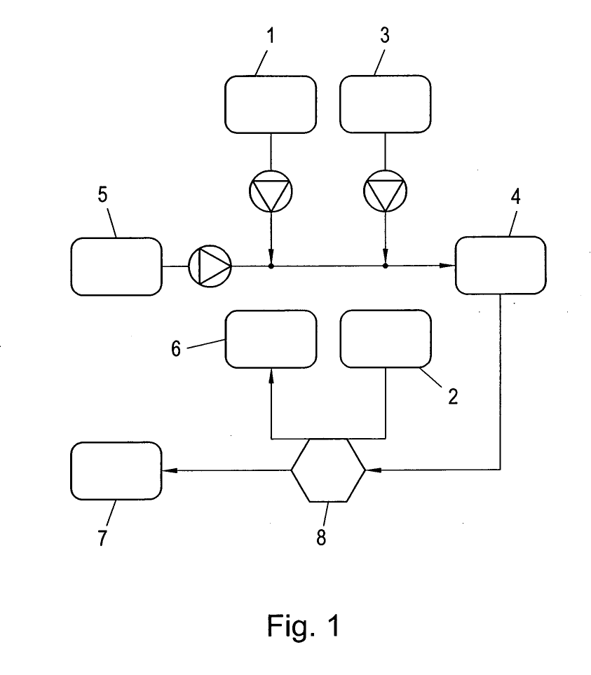

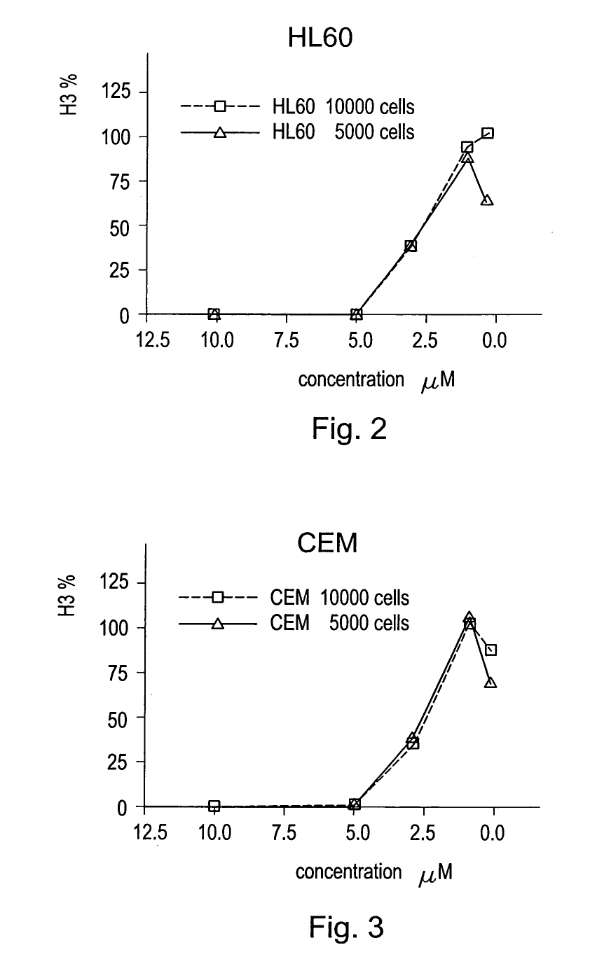

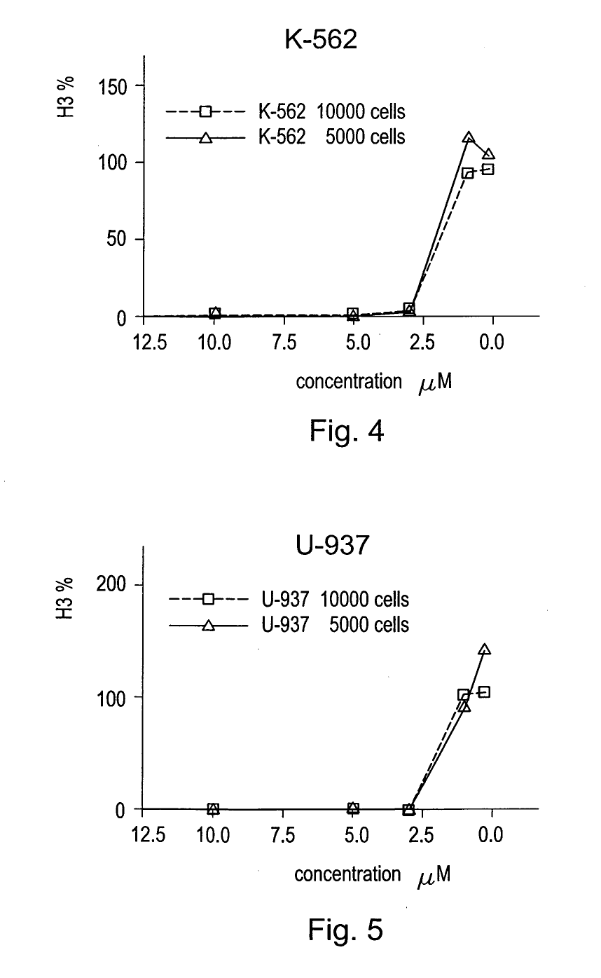

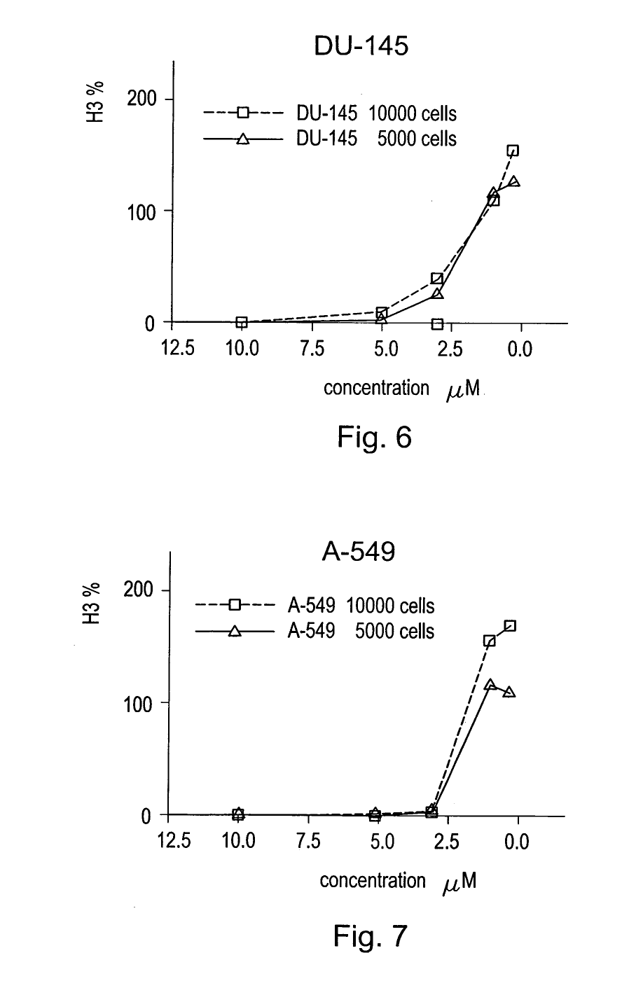

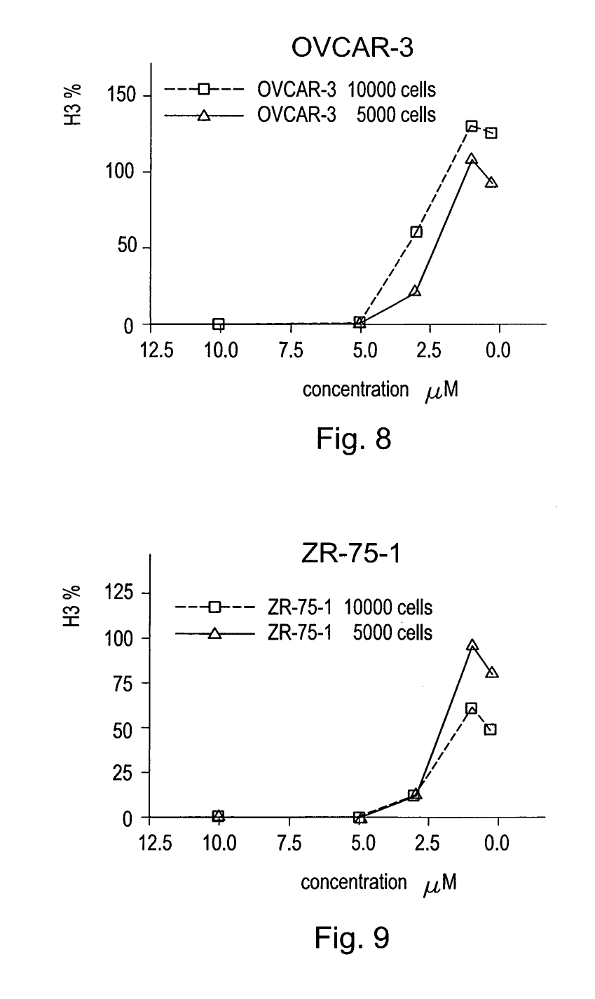

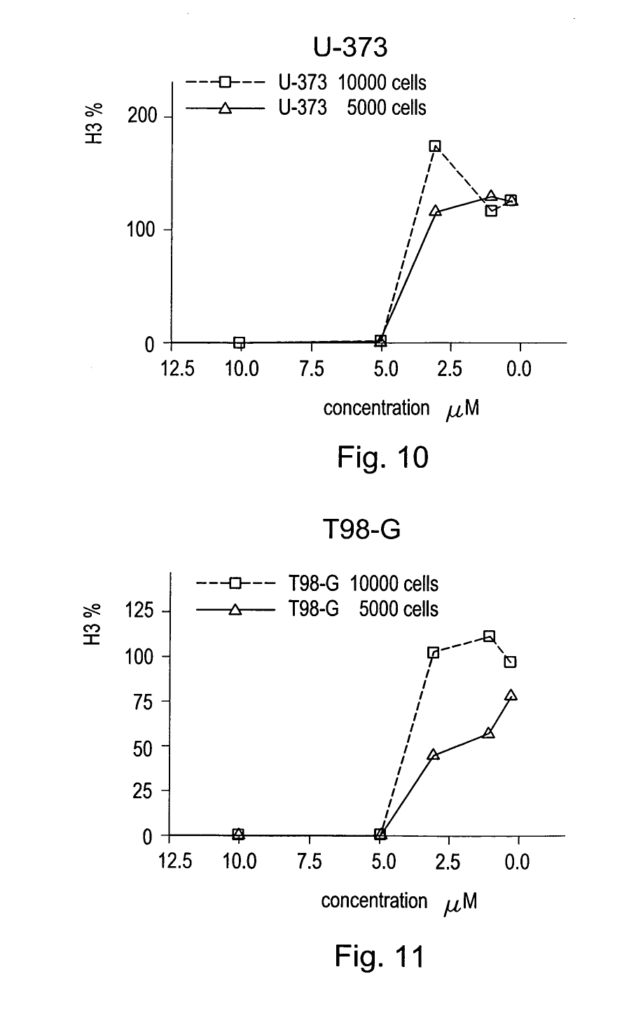

### EXAMPLE 1

### Liposomal Formulation of poly-[2-(2-ethoxyethoxyethyl)guanidinium hydrochloride]

Poly-[2-(2-ethoxyethoxyethyl)guanidinium hydrochloride] has been encapsulated in liposomes by the modified ethanol injection system as described above using the process parameters summarized in Table 8. By using the two lipids DMPC and DSPE-PEG-2000, the API is encapsulated in a PEGylated liposomal formulation, since DSPE-PEG-2000 will form a PEGylated outer layer of the liposomal micellar bilayer. Resulting liposomes revealed sizes between 100 and 130 nm with PdIs between 0.1 and 0.2 and inclusion rates of the active ingredient around 15 to 20%. For size and PdI data see Table 9 below.

TABLE 8

| Aqueous phases + | 190 ml injection buffer |
|---|---|
| volumes | (39% API in |
| | phys. NaCl) |
| | 760 ml dilution |
| | buffer (PBS) |
| Injection module diameter | 350 μm |
| Ethanol concentration in intermediate liposome | 5% |
| suspension | |
| Injection pressure | 5 bar |
| Lipid concentration in intermediate solution | 28.5/1.5 |
| DMPC/DSPE-PEG-2000 [μmol/ml] | |
| AMF 1000 concentration | 39% |
| Temperature ethanol solution/aqueous phase | 55° C. |
| Diafiltration | 15-20 volume changes |
| Ultrafiltration: concentration factor | 8 |

Table 8 shows process parameters.

TABLE 9

| | Size [nm] | PdI |
|---|---|---|
| Poly-[2-(2-ethoxyethoxyethyl)guanidinium | 121.4 | 0.143 |
| hydrochloride] in mPEG2000-liposomes, batch 1 | | |
| Poly-[2-(2-ethoxyethoxyethyl)guanidinium | 121.4 | 0.143 |
| hydrochloride] in mPEG2000-liposomes, batch 2 | | |
| Poly-[2-(2-ethoxyethoxyethyl)guanidinium | 124.5 | 0.137 |
| hydrochloride] in mPEG2000-liposomes, batch 3 | | |

Table 9 shows size- and PdI-data for poly-[2-(2-ethoxyethoxyethyl)guanidinium hydrochloride] encapsulated in PEGylated liposomes.

### EXAMPLE 2

### Liposomal Formulation of Polyhexamethylenguanidinium Hydrochloride

Polyhexamethylenguanidinium hydrochloride has been encapsulated in PEGylated liposomes in the same way and with the identic process parameters as set out in Table 8 for poly-[2-(2-ethoxyethoxyethyl)guanidinium hydrochloride] and revealed a little bit smaller liposomes in sizes around 80 to 100 nm, presumably caused by the smaller size of the encapsulated molecule. For size- and PdI-data see Table 10 below.

TABLE 10

| | Size [nm] | PdI |
|---|---|---|
| Polyhexamethylenguanidinium hydrochloride in | 88.1 | 0.130 |
| mPEG2000-liposomes, batch 1 | | |
| Polyhexamethylenguanidinium hydrochloride in | 103.4 | 0.182 |
| mPEG2000-liposomes, batch 2 | | |
| Polyhexamethylenguanidinium hydrochloride in | 95.95 | 0.212 |
| mPEG2000-liposomes, batch 3 | | |

Table 10 shows size- and PdI-data for polyhexamethylenguanidinium hydrochloride encapsulated in PEGylated liposomes.

### EXAMPLE 3

### Liposomal Formulation of poly-[2-(2-ethoxyethoxyethyl)guanidinium hydrochloride]- and poly-[hexamethylengaunidinium hydrochloride]-copolymer

Poly-[2-(2-ethoxyethoxyethyl)guanidinium hydrochloride- and poly-[hexamethylengaunidinium hydrochloride]-copolymer was the third candidate of polymeric guanidine derivates to be encapsulated in PEGylated liposomes. Again the same process parameters as used for the encapsulation of poly-[2-(2-ethoxyethoxyethyl)guanidinium hydrochloride] (see Table 8) have been applied. As could be expected the experiment resulted in similar liposomes as the two investigated polymeric guanidine derivates before. For size- and PdI-data see Table 11 below.

TABLE 11

| | Size | |
|---|---|---|
| | [nm] | PdI |
| Poly-[2-(2-ethoxyethoxyethyl)guanidinium hydrochloride- | 101.9 | 0.121 |
| and poly-[hexamethylengaunidinium hydrochloride]- | | |
| copolymer in mPEG2000-liposomes, batch 1 | | |
| Poly-[2-(2-ethoxyethoxyethyl)guanidinium hydrochloride- | 103.7 | 0.142 |
| and poly-[hexamethylengaunidinium hydrochloride]- | | |
| copolymer in mPEG2000-liposomes, batch 2 | | |
| Poly-[2-(2-ethoxyethoxyethyl)guanidinium hydrochloride- | 118.6 | 0.130 |
| and poly-[hexamethylengaunidinium hydrochloride]- | | |
| copolymer in mPEG2000-liposomes, batch 3 | | |

Table 11 shows size- and PdI-data for poly-[2-(2-ethoxyethoxyethyl)guanidinium hydrochloride- and poly-[hexamethylengaunidinium hydrochloride]-copolymer encapsulated in PEGylated liposomes.

Beside the substances exemplified in examples 1 to 3 the present invention includes inter alia also substances such as polymeric biguanidines and other polymeric guanidine derivates.

The activity of the liposomal formulations according to the present invention as described in examples 1, 2 and 3 has been tested in-vitro in different concentrations and in several cell lines using an H-thymidin-test as described above. However, the drug formulation described in example 1 has shown improved tolerability also in in-vivo models.

A tolerance study conducted in mice showed that in contrast to the free API the liposomal encapsulated formulation according to the present invention is tolerated at a daily intravenous dosing regime at a dose of 2.5 mg/kg body weight. On a weekly basis even 5 mg/kg body weight have been well tolerated. Whereas non-liposomal formulations of polymeric guanidine derivates induce necrosis e.g. in the tail vein, this is not the case with the PEGylated liposomal formulation according to the present invention, which is the proof that the active ingredient is not accumulated at the injection site, but systemically distributed by the blood stream.

Based on this intravenous tolerance study in mice, a clinical case study with a half-breed dog (Husky—German Shepherd dog) suffering from hemangiosarcoma Stage III (T2N0M1) with multiple lung metastases was conducted. The terminal clinical state of the patient and the request of his holder allowed for a therapy with the provided drug—liposomal formulated poly-[2-(2-ethoxyethoxyethyl)guanidinium hydrochloride] at a veterinary university center—in line with present scientific knowledge and the therapeutic possibilities in this disease and therefore is equivalent to a compassionate treatment attempt according to the declaration of Helsinki in human medicine and is ethically justifiable.

The dog was treated three times—on day 1, 3 and 8—with a dose of 5 mg/kg body weight diluted in physiological sodium chloride solution by intravenous infusion. The therapy was well tolerated and the dog did not show clinical signs of side effects and the blood counts were not affected by the therapy. Two weeks after start of the therapy the radiological control showed a disease stabilization of the lung lesions compared to the baseline CT-examination. The observed effect was accompanied by an improved clinical state and situation. Another important fact is that the white and red blood cells did not show a significant decline like under cytostatic therapy, which can be attributed to the passive targeting properties of the liposomal formulation presented in this invention as set out in Table 12 below.

TABLE 12

| AMF 1000 liposomal | Day 1 | Day 3 | | | Day 14 | Day 15 |
|---|---|---|---|---|---|---|
| (Dose) | 200 mg | 200 mg | Day 8 | Day 13 | 75 mg | 75 mg |
| Erythrocytes | 4.91 | 4.89 | 4.84 | 4.22 | | 3.81 |
| Haematocrit | 34.6 | 35 | 34.8 | 31.4 | | 28.3 |
| Retikulocytes abs. | | 102323 | 257972 | 468420 | | |
| Leukocyte | 10320 | 11390 | 14700 | 20060 | | 16960 |
| band neutrophil | 0.1 | 0.11 | 0.15 | 0.2 | | 0.17 |
| segmented neutrophil | 6119.76 | 7745.2 | 10672 | 17853 | | 13534 |
| Lymphocyte | 2404.56 | 2164.1 | 2160.9 | 802.4 | | 1526 |
| Monocyte | 474.72 | 683.4 | 823.2 | 1203.6 | | 1018 |
| Eosinophile | 1279.68 | 797.3 | 940.8 | 200.6 | | 746.2 |
| Basophile | 20.64 | 0.11 | 14.7 | 0.2 | | 33.92 |
| Thrombocyte | 149 | 124 | 149 | 188 | | 105 |
| Albumin | 3.95 | | 3.72 | 3.76 | | |
| Total protein | 6.44 | 5.88 | 6.36 | 6.46 | | 5.63 |
| Urea | 52.2 | 25.1 | 54.9 | 36 | | |
| Creatinine | 1 | 1 | 1.7 | 1.5 | | 1.6 |
| Alkaline phosphatase | 59 | 52 | | 36 | | |
| ALT | 60 | 101 | 52 | 38 | | |
| LDH | 110 | 128 | 80 | 90 | | |
| Creatine kinase | 298 | 4353 | 196 | 154 | | 946 |
| Potassium | 4.2 | 3.9 | 4.1 | 3.4 | | |
| Calcium | 2.77 | 2.62 | 2.65 | 2.65 | | |
| Phosphor | 1.4 | 1.33 | | 1.14 | | |
| Prothrombin time (PT) | 12.5 | | | | | 10.6 |
| Partial Thromboplastin Time (PTT) | 15.8 | | | | | 17.7 |
| Thrombin time (TT) | 18.1 | | | | | 17.1 |

| AMF 1000 liposomal | Day 16 | Day 17 | | | | |
|---|---|---|---|---|---|---|
| (Dose) | 75 mg | 75 mg | Day 20 | Unit | Reference range | Dev. |
| Erythrocytes | | 4.09 | 4.25 | 10E6/μl | 5.50-8.00 | − |
| Haematocrit | | 30.6 | 31.5 | % | 37.00-55.00 | − |
| Retikulocytes abs. | | | | /μl | >60000 | |
| Leukocyte | | 16960 | 12310 | /μl | 6000.0-15000.0 | + |
| band neutrophil | | 0.25 | 0.12 | /μl | <500.00 | |
| segmented neutrophil | | 20605 | 9872.6 | /μl | 3300.00-11250.00 | |
| Lymphocyte | | 1862 | 1267.9 | /μl | 780.00-4500.00 | |
| Monocyte | | 1446 | 541.64 | /μl | <500.00 | + |
| Eosinophile | | 343 | 553.95 | /μl | <800.00 | + |
| Basophile | | 24.5 | 36.93 | /μl | <150.00 | |
| Thrombocyte | | 201 | 201 | 10E3/μl | 150-500 | − |
| Albumin | | 3.57 | 3.68 | g/dl | 2.58-4.73 | |
| Total protein | | 6.18 | 6.3 | g/dl | 6.00-7.5 | − |
| Urea | | 27.2 | 41.1 | mg/dl | 20.0-40.0 | + |
| Creatinine | | 1.8 | 2 | mg/dl | 0.40-1.20 | + |
| Alkaline phosphatase | | | 24 | U/L | <130 | |
| ALT | | 35 | 29 | U/L | <80 | + |
| LDH | | | 86 | U/L | <60 | + |
| Creatine kinase | | | 149 | U/L | <250 | + |
| Potassium | | 4.4 | | | | |
| Calcium | | | | | | |
| Phosphor | | 1.91 | 1.36 | mmol/L | 0.90-1.60 | + |
| Prothrombin time (PT) | | | | Sek. | 8.0-10.0 | + |
| Partial Thromboplastin Time (PTT) | | | | Sek. | 8.0-15.0 | + |
| Thrombin time (TT) | | | | Sek. | <20.0 | |

Dev., Deviations;
Retikulocytes abs., Retikulocytes absolute;
ALT, alanine aminotransferase;
LDH, lactatedehydrogenase
Table 12 above shows blood counts before and during therapy with the liposomal drug composition.

Further infusions were given on a daily basis on days 14 to 17 with a dose of 2 mg/kg body weight. By then the dog had survived for more than 30 days despite an initial prognosis at treatment start of a few days. After the therapies the dog showed a good clinical condition and regained its normal activity.

The drug composition according to the present invention appears to be relatively well tolerated and induced a disease stabilization in this terminally ill dog suffering from a far progressed hemangiosarcoma with multiple lung lesions, little to no hematological and organ toxicity was observed.

### ABBREVIATIONS

API active pharmaceutical ingredient (in this patent application a polymeric guanidine derivative encapsulated in liposomes according to the present invention.)
DMPC 1,2-dimyristoyl-sn-glycero-3-phosphocholine
DPPC 1,2-dipalmitoyl-sn-glycero-3-phosphocholine
DMPG 1,2-dimyristoyl-sn-glycero-3-phospho-(1′-rac-glycerol)
DSPE-PEG 2000 1,2-distearoyl-sn-glycero-3-phosphoethanolamine-N—[amino(polyethylene glycol)-2000]
PBS Phosphate buffered saline
PES Polyethersulfone
PdI Polydispersity Index
PEG Polyethylenglycol

1. A liposomal drug composition comprising:
a dimeric or polymeric guanidine derivative or a pharmaceutically acceptable salt thereof as drug substance; and
a lipid modified by polyethylene glycole (PEG).

2. A liposomal drug composition according to claim 1, wherein the lipid is a phospholipid and said PEG is PEG₅₀₀-PEG₅₀₀₀.

3. A liposomal drug composition according to claim 1, wherein said polymeric guanidine derivative is one, which guanidine derivative is based on a diamine containing oxyalkylene chains between two amino groups, with the guanidine derivative representing a product of polycondensation between a guanidine acid addition salt and a diamine containing polyoxyalkylene chains between two amino groups.

4. A liposomal drug composition according to claim 3, wherein among the representatives of the family of polyoxyalkylene guanidine salts, there are such using triethylene glycol diamine (relative molecular mass: 148), polyoxypropylene diamine (relative molecular mass: 230) as well as polyoxyethylene diamine (relative molecular mass: 600).

5. A liposomal drug composition according to claim 1, wherein poly-[2-(2-ethoxyethoxyethyl)guanidinium hydrochloride] comprising at least 3 guanidinium groups is contained as the drug substance.

6. A drug composition according to claim 5, wherein the average molecular mass of the drug substance ranges from 500 to 3000.

7. A method of preparing a cytostatically active liposomal drug composition using the dimeric or polymeric guanidine derivative of claim 1.

8. A method of preparing an antimicrobial drug composition using the dimeric or polymeric guanidine derivative of claim 1.

9. A process for therapeutically treating human beings and animals, wherein the drug composition according to claim 1 is injected into a human being or an animal in need thereof.

10. A liposomal drug composition according to claim 2, wherein said polymeric guanidine derivative is one, which guanidine derivative is based on a diamine containing oxyalkylene chains between two amino groups, with the guanidine derivative representing a product of polycondensation between a guanidine acid addition salt and a diamine containing polyoxyalkylene chains between two amino groups.

11. A liposomal drug composition according to claim 10, wherein among the representatives of the family of polyoxyalkylene guanidine salts, there are such using triethylene glycol diamine (relative molecular mass: 148), polyoxypropylene diamine (relative molecular mass: 230) as well as polyoxyethylene diamine (relative molecular mass: 600).

12. A liposomal drug composition according to claim 2, wherein poly-[2-(2-ethoxyethoxyethyl)guanidinium hydrochloride] comprising at least 3 guanidinium groups is contained as the drug substance.

13. A drug composition according to claim 12, wherein the average molecular mass of the drug substance ranges from 500 to 3000.

14. A liposomal drug composition according to claim 3, wherein poly-[2-(2-ethoxyethoxyethyl)guanidinium hydrochloride] comprising at least 3 guanidinium groups is contained as the drug substance.

15. A drug composition according to claim 14, wherein the average molecular mass of the drug substance ranges from 500 to 3000.

16. A liposomal drug composition according to claim 4, wherein poly-[2-(2-ethoxyethoxyethyl)guanidinium hydrochloride] comprising at least 3 guanidinium groups is contained as the drug substance.

17. A drug composition according to claim 16, wherein the average molecular mass of the drug substance ranges from 500 to 3000.

18. A process for therapeutically treating human beings and animals, wherein the drug composition according to claim 3 is injected into a human being or an animal in need thereof.

19. A process for therapeutically treating human beings and animals, wherein the drug composition according to claim 4 is injected into a human being or an animal in need thereof.

20. A process for therapeutically treating human beings and animals, wherein the drug composition according to claim 5 is injected into a human being or an animal in need thereof.
